(19) United States
(12) Patent Application Publication
(10) Pub. No.: US 2026/0245244 A1
(43) Pub. Date: Aug. 20, 2026

(54) REPRESENTING A QUANTITATIVE PROPERTY OF AN ANATOMICAL OBJECT IN A USER INTERFACE

(71) Applicant: Intuitive Surgical Operations, Inc., Sunnyvale, CA (US)

(72) Inventors: Dennise D. Dalma-Weiszhausz, Palo Alto, CA (US); Jeffrey M. DiCarlo, Austin, TX (US); Abhishek Bichal, San Jose, CA (US); Jennifer Bittner, Santa Clara, CA (US); Simon P. DiMaio, San Carlos, CA (US); Brandon D. Itkowitz, San Jose, CA (US); Sarah Johnson, San Jose, CA (US); Anshul Malhan, Mountain View, CA (US); Theodore W. Rogers, Alameda, CA (US); David Van Story, San Francisco, CA (US); Lin Yue, Fremont, CA (US); Sarthak Ghosh, Sunnyvale, CA (US)

(21) Appl. No.: 19/153,711

(22) PCT Filed: Feb. 29, 2024
(86) PCT No.: PCT/US2024/017860

§ 371 Date: Aug. 5, 2025

Related U.S. Application Data
(60) Provisional application No. 63/449,384, filed on Mar. 2, 2023.

(51) Int. Cl. G06T 7/73; A61B 1/00; A61B 1/04; G06T 7/00; G06V 10/25; G06V 10/70; G16H 40/63

(52) U.S. Cl. CPC G06T 7/74; A61B 1/00039; A61B 1/00045; A61B 1/043; G06T 7/0014; G06V 10/25; G06V 10/70; G16H 40/63; G06T 2200/24; G06T 2207/10024; G06T 2207/10064; G06T 2207/20104; G06T 2207/30004; G06V 2201/03

(57) ABSTRACT

An illustrative property value system may be configured to provide, for display in a user interface, an image of an anatomical scene as captured by an imaging device and identify a reference location and an additional location in the image of the anatomical scene. The system may further be configured to determine a value of a property of an anatomical object at the additional location relative to the reference location and cause display, in the user interface together with the image of the anatomical scene, of output data representative of the reference location, the additional location, and the value of the property of the anatomical object at the additional location relative to the reference location.

### RELATED APPLICATIONS

This application claims priority to U.S. Provisional Patent Application No. 63/449,384, filed Mar. 2, 2023, the content of which is hereby incorporated by reference in its entirety.

### BACKGROUND INFORMATION

During a medical procedure, an imaging device (e.g., an endoscope) may be used to capture images of a patient's internal anatomy. The images may be presented (e.g., in the form of a video stream) to a surgeon during the medical procedure to assist the surgeon in performing the medical procedure. In some instances, it may be desirable to assess a property (e.g., fluorescence, oxygen saturation, temperature, potential hydrogen (pH), tissue-specific binding, etc.) of the patient's internal anatomy based on the images depicting the anatomy.

As an illustrative example, the images may include or be augmented with fluorescence images. For this, one or more fluorescent agents (e.g., indocyanine green (ICG) dye) may be administered to the patient such that the fluorescence images may be generated based on detected fluorescence emitted by the one or more fluorescent agents at particular areas of the anatomy when the one or more fluorescent agents are excited by fluorescence excitation illumination. In some instances, the detected fluorescence may be highlighted in the fluorescence images with a selected color (e.g., green).

In some scenarios, the fluorescence images may be used to correlate an intensity of the selected color depicted in the fluorescence images with an amount of fluorescence emitted at particular areas of the anatomy (e.g., by the fluorescent agent). However, such correlations may depend on a number of factors (e.g., a distribution of fluorescence excitation illumination applied to the anatomy, a position of a light source providing the fluorescence excitation illumination, a depth of the fluorescent agent within the anatomy, etc.), which may cause the correlations to be subjective and/or inconsistent.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An illustrative system includes a memory storing instructions and a processor communicatively coupled to the memory and configured to execute the instructions to perform a process comprising: causing display, in a user interface during a medical procedure, of an image of an anatomical scene captured by an imaging device during the medical procedure; identifying a reference in the image of the anatomical scene; identifying an additional location in the image of the anatomical scene; determining a fluorescence value representative of a normalized amount of fluorescence emitted at the additional location relative to a normalized amount of fluorescence emitted at the reference location; and causing display, in the user interface together with the image of the anatomical scene during the medical procedure, of output data representative of the reference location, the additional location, and the fluorescence value.

An illustrative method includes: causing display, by at least one computing device and in a user interface during a medical procedure, an image of an anatomical scene captured by an imaging device during the medical procedure; identifying, by the at least one computing device, a reference location in the image of the anatomical scene; identifying, by the at least one computing device, an additional location in the image of the anatomical scene; determining, by the at least one computing device, a fluorescence value representative of a normalized amount of fluorescence emitted at the additional location relative to a normalized amount of fluorescence emitted at the reference location; and causing display, by the at least one computing device and in the user interface together with the image of the anatomical scene during the medical procedure, of output data representative of the reference location, the additional location, and the fluorescence value.

An illustrative non-transitory computer-readable medium may store instructions that, when executed, direct a processor of a computing device to: cause display, in a user interface during a medical procedure, an image of an anatomical scene captured by an imaging device during the medical procedure; identify a reference location in the image of the anatomical scene; identify an additional location in the image of the anatomical scene; determine a fluorescence value representative of a normalized amount of fluorescence emitted at the additional location relative to a normalized amount of fluorescence emitted at the reference location; and cause display, in the user interface together with the image of the anatomical scene during the medical procedure, of output data representative of the reference location, the additional location, and the fluorescence value.

An illustrative system includes a memory storing instructions and a processor communicatively coupled to the memory and configured to execute the instructions to perform a process comprising: identifying a region of interest in an anatomical scene; determining values of a property of an anatomical object depicted at positions in the region of interest; determining, based on the values of the property of the anatomical object, a transition position representative of a position in the region of interest at which at least some of the values of the property transition across a property value threshold; and causing a display device to display output data representative of the transition position in the region of interest.

An illustrative method includes: identifying, by at least one computing device, a region of interest in an anatomical scene; determining, by the at least one computing device, values of a property of an anatomical object depicted at positions in the region of interest; determining, by the at least one computing device and based on the values of the property of the anatomical object, a transition position representative of a position in the region of interest at which at least some of the values of the property transition across a property value threshold; and causing, by the at least one computing device, a display device to display output data representative of the transition position in the region of interest.

An illustrative non-transitory computer-readable medium may store instructions that, when executed, direct a processor of a computing device to: identify a region of interest in an anatomical scene; determine values of a property of an anatomical object depicted at positions in the region of interest; determine, based on the values of the property of the anatomical object, a transition position representative of a position in the region of interest at which at least some of the values of the property transition across a property value threshold; and cause a display device to display output data representative of the transition position in the region of interest.

An illustrative system includes a memory storing instructions and a processor communicatively coupled to the memory and configured to execute the instructions to perform a process comprising: determining, based on a reference location identified in an anatomical scene, one or more property values representative of a value of a property of an anatomical object at one or more additional locations spaced a select distance away from the reference location relative to a value of the property of the anatomical object at the reference location; and causing a display device to display output data representative of the one or more property values.

An illustrative method includes: determining, by at least one computing device and based on a reference location identified in an anatomical scene, one or more property values representative of a value of a property of an anatomical object at one or more additional locations spaced a select distance away from the reference location relative to a value of the property of the anatomical object at the reference location; and causing, by the at least one computing device, a display device to display output data representative of the one or more property values.

An illustrative non-transitory computer-readable medium may store instructions that, when executed, direct a processor of a computing device to: determine, based on a reference location identified in an anatomical scene, one or more property values representative of a value of a property of an anatomical object at one or more additional locations spaced a select distance away from the reference location relative to a value of the property of the anatomical object at the reference location; and cause a display device to display output data representative of the one or more property values.

An illustrative system includes a memory storing instructions and a processor communicatively coupled to the memory and configured to execute the instructions to perform a process comprising: causing display, in a user interface during a medical procedure, of an image of an anatomical scene as captured by an imaging device during the medical procedure; identifying a reference location in the image of the anatomical scene; identifying an additional location in the image of the anatomical scene; determining a value of a property of an anatomical object at the additional location relative to the reference location; and causing display, in the user interface together with the image of the anatomical scene during the medical procedure, of output data representative of the reference location, the additional location, and the value of the property of the anatomical object at the additional location relative to the reference location.

An illustrative method includes: causing display, by at least one computing device and in a user interface during a medical procedure, of an image of an anatomical scene as captured by an imaging device during the medical procedure;

identifying, by the at least one computing device, a reference location in the image of the anatomical scene; identifying, by the at least one computing device, an additional location in the image of the anatomical scene; determining, by the at least one computing device, a value of a property of an anatomical object at the additional location relative to the reference location; and causing display, by the at least one computing device and in the user interface together with the image of the anatomical scene during the medical procedure, of output data representative of the reference location, the additional location, and the value of the property of the anatomical object at the additional location relative to the reference location.

An illustrative non-transitory computer-readable medium may store instructions that, when executed, direct a processor of a computing device to: cause display, in a user interface during a medical procedure, of an image of an anatomical scene as captured by an imaging device during the medical procedure; identify a reference location in the image of the anatomical scene; identify an additional location in the image of the anatomical scene; determine a value of a property of an anatomical object at the additional location relative to the reference location; and cause display, in the user interface together with the image of the anatomical scene during the medical procedure, of output data representative of the reference location, the additional location, and the value of the property of the anatomical object at the additional location relative to the reference location.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 depicts an illustrative implementation including a property value system according to principles described herein.

FIG. 2 depicts an illustrative method of operating a property value system according to principles described herein.

FIG. 3 shows an illustrative implementation of a fluorescence imaging system for use with a property value system according to principles described herein.

FIG. 4 depicts another illustrative method of operating a property value system according to principles described herein.

FIG. 5 depicts an illustrative implementation of a user interface according to principles described herein.

FIG. 6 depicts another illustrative implementation of a user interface according to principles described herein.

FIG. 7 depicts another illustrative implementation of a user interface according to principles described herein.

FIG. 8 depicts another illustrative method of operating a property value system according to principles described herein.

FIG. 9 depicts another illustrative method of operating a property value system according to principles described herein.

FIG. 10 depicts an illustrative computing system according to principles described herein.

FIG. 11 depicts an illustrative implementation of a computer-assisted medical system according to principles described herein.

### DETAILED DESCRIPTION

An illustrative property value system may be configured to represent a quantitative property (e.g., fluorescence, oxygen saturation, temperature, pH, tissue-specific binding, etc.) of an anatomical object in a user interface.

In certain implementations, for example, the property value system may be configured to cause display, in a user interface, of an image of an anatomical scene (e.g., as captured by an imaging device during a medical procedure), identify a reference location in the image, identify an additional location in the image, determine a value of a property of an anatomical object at the additional location relative to the reference location, and cause display, in the user interface together with the image of the anatomical scene, of output data representative of the reference location, the additional location, and the value of the property of the anatomical object.

To illustrate, the property value system may be configured to determine a fluorescence value representative of a normalized amount of fluorescence emitted at the additional location relative to a normalized amount of fluorescence emitted at the reference location such that the output data may include the fluorescence value. Additionally or alternatively, the property value system may determine an oxygen saturation value representative of an amount of oxygen saturation of the anatomical object at the additional location relative to an amount of oxygen saturation of the anatomical object at the reference location. Still values of other properties (e.g., temperature, pH, tissue-specific binding, etc.) of the anatomical object may be determined by the property value system.

In some embodiments, the property value system may be configured to detect a user input designating the reference location and/or the additional location in the image of the anatomical scene. The additional location may include one or more points and/or a region of interest in the image of the anatomical scene. In some instances, the additional location may be spaced a select distance away from the reference location. The property value system may further be configured to determine, based on the property of the anatomical object at the additional location relative to the reference location, a transition position representative of a position in the region of interest at which at least some of the values of the property transition across a property value threshold.

The principles described herein may result in an improved representation of a property of an anatomical object compared to conventional techniques that are not based on quantitative values, as well as provide other benefits as described herein. For example, providing a quantitative property value of an anatomical object for display in a user interface may allow the property value to be determined by a user objectively, consistently, and/or accurately. Additionally, a user may interact with the user interface to designate the reference location and/or additional location, which may allow the quantitative property value representations to be customizable.

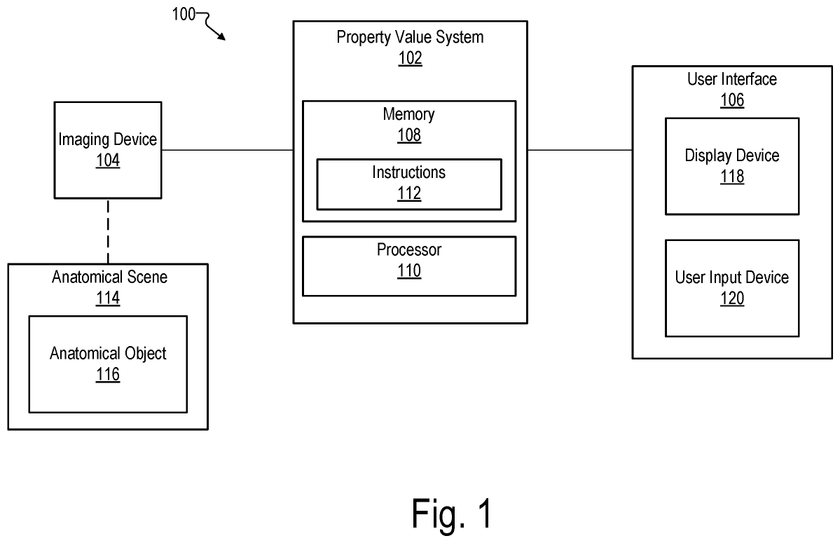

FIG. 1 shows an illustrative implementation 100 configured to provide a representation of a quantitative property of an anatomical object. As shown, implementation 100 includes a property value system 102 communicatively coupled (e.g., wired and/or wirelessly) with an imaging device 104 and a user interface 106. Implementation 100 may include additional or alternative components as may serve a particular implementation. In some examples, components of property value system 102, imaging device 104, and/or user interface 106 may be implemented by a computer-assisted medical system.

Property value system 102 may be implemented by one or more computing devices and/or computer resources (e.g., processors, memory devices, storage devices, etc.) as may serve a particular implementation. As shown, property value system 102 may include, without limitation, a memory 108 and a processor 110 selectively and communicatively coupled to one another. Memory 108 and processor 110 may each include or be implemented by computer hardware that is configured to store and/or process computer software. Various other components of computer hardware and/or software not explicitly shown in FIG. 1 may also be included within property value system 102. In some examples, memory 108 and/or processor 110 may be distributed between multiple devices and/or multiple locations as may serve a particular implementation.

Memory 108 may store and/or otherwise maintain executable data used by processor 110 to perform any of the functionality described herein. For example, memory 108 may store instructions 112 that may be executed by processor 110. Memory 108 may be implemented by one or more memory or storage devices, including any memory or storage devices described herein, that are configured to store data in a transitory or non-transitory manner. Instructions 112 may be executed by processor 110 to cause property value system 102 to perform any of the functionality described herein. Instructions 112 may be implemented by any suitable application, software, code, and/or other executable data instance. Additionally, memory 108 may also maintain any other data accessed, managed, used, and/or transmitted by processor 110 in a particular implementation.

Processor 110 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 110 (e.g., when processor 110 is directed to perform operations represented by instructions 112 stored in memory 108), property value system 102 may perform various operations as described herein.

Imaging device 104 may be implemented by an endoscope or other device(s) configured to capture an anatomical scene 114 (e.g., a three-dimensional (3D) scene or a two-dimensional (2D) scene). In some implementations, imaging device 104 may include video imaging devices, infrared imaging devices, visible light imaging devices, non-visible light imaging devices, intensity imaging devices (e.g., color, grayscale, black and white imaging devices), depth imaging devices (e.g., stereoscopic imaging devices, time-of-flight imaging devices, infrared imaging devices, red-green-blue (RGB) imaging devices, red-green-blue and depth (RGB-D) imaging devices, light detection and ranging (LIDAR) imaging devices, etc.), any other imaging devices, or any combination or sub-combination of such imaging devices.

Imaging device 104 may be positioned relative to anatomical scene 114 and may be configured to image anatomical scene 114. In some implementations, imaging device 104 may be moved relative to anatomical scene 114 to image anatomical scene 114 at different viewpoints. As used herein, an “image” may include a video stream and/or one or more still image snapshots. In some implementations, the images may include image data (e.g., color, grayscale, saturation, intensity, brightness, depth, etc.) captured by imaging device 104. The image data may, in some instances, be associated with data points expressed in a common coordinate frame such as 3D voxels or 2D pixels of images captured by imaging device 104. Imaging device 104 may be configured to capture images of anatomical scene 114 at any suitable capture rates.

An anatomical scene 114 may include an environment (e.g., an area within a subject of a medical procedure) and/or one or more objects within an environment. For example, anatomical scene 114 may include an anatomical object 116. Anatomical object 116 may include an object associated with a subject (e.g., a body of a live animal, a human or animal cadaver, a portion of human or animal anatomy, tissue removed from human or animal anatomies, non-tissue work pieces, training models, etc.). In some implementations, anatomical object 116 may include tissue of a subject (e.g., an organ, soft tissue, connective tissue, etc.).

User interface 106 of the illustrated implementation comprises a display device 118 and a user input device 120. Display device 118 may be implemented by a monitor or other suitable device configured to display information to a user. For example, display device 118 may be configured to display an image or other information based on anatomical scene 114 captured by imaging device 104. User input device 120 may be implemented by any suitable device or devices (e.g., a button, joystick, touchscreen, keyboard, handle, microphone, etc.) configured to receive a user input, for example, to interact with the display presented by display device 118.

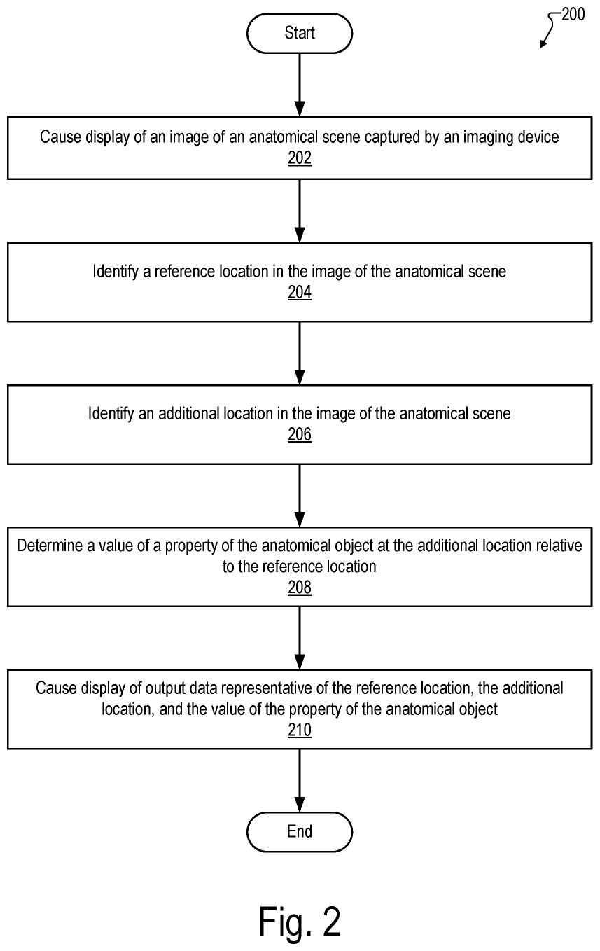

FIG. 2 shows an illustrative method 200 that may be performed by property value system 102. While FIG. 2 illustrates example operations according to one embodiment, other embodiments may omit, add to, reorder, and/or modify any of the operations shown in FIG. 2. Moreover, each of the operations depicted in FIG. 2 may be performed in any of the ways described herein.

As shown, property value system 102 may, at operation 202, cause display of an image of anatomical scene114 as captured by imaging device 104. The causing display of the image may include one or more operations of property value system 102. For example, the causing display of the image may include transmitting the image to user interface 106 for display by display device 118. In some implementations, the causing display of the image may further include property value system 102 performing a sequence of operations to provide the image. For example, property value system 102 may receive an image of anatomical scene 114 captured by imaging device 104, process the received image, and provide the processed image for display by user interface 106. As an illustrative example, property value system 102 may be configured to fuse images of anatomical scene 114 captured by imaging device 104 at different viewpoints of anatomical scene 114. In certain examples, the fusing may include merging aligned (or overlapping) voxels or pixels, such as by blending intensity and/or depth values for aligned voxels or pixels. The fusing may additionally or alternatively include stitching non-overlapping voxels or pixels together, such as by stitching images together along non-overlapping boundaries of the images.

In some implementations, the image may be captured by imaging device 104 during a medical procedure (e.g., a surgical procedure, a diagnostic procedure, a biopsy procedure, etc.) such that property value system 102 may provide the image for display in user interface 106 during the medical procedure. In some instances, the medical procedure may include a minimally-invasive procedure.

In some implementations, the image may be provided by property value system 102 in real time. For example, the image may be transmitted (e.g., in the form of a video stream) by property value system 102 to user interface 106 as the image is received and/or processed by property value system 102. This may allow the image to be displayed by display device 118 of user interface 106 during the medical procedure (e.g., as the fluorescent agent flows through anatomical object 116).

Property value system 102 may, at operation 204, identify a reference location in the image of anatomical scene 114 (e.g., associated with anatomical object 116). Property value system 102 may further, at operation 206, identify an additional location in the image of anatomical scene 114 (e.g., associated with anatomical object 116). For example, the identifying the reference location and/or the additional location may include identifying one or more points (e.g., one or more pixels and/or voxels forming discrete locations) in the image of anatomical scene 114. Additionally or alternatively, the identifying the reference location and/or the additional location may include identifying a region of interest (e.g., an area of a group of pixels and/or voxels) in the image of anatomical scene 114.

In some implementations, the identifying the reference location and/or the additional location may include detecting a user input designating the reference location and/or the additional location. For example, the image of anatomical scene 114 provided by property value system 102 may be displayed by display device 118 of user interface 106. A user may interact with user input device 120 to designate the reference location and/or the additional location such as by a discrete event (e.g., a touch gesture, a button press, a mouse click, a button release, etc.) on any point of the image of anatomical scene 114 on display device 118.

Additionally or alternatively, the identifying the reference location and/or the additional location may include implementing and applying artificial intelligence algorithms, such as machine learning algorithms, to designate the reference location and/or the additional location. Any suitable form of artificial intelligence and/or machine learning may be used, including, for example, deep learning, neural networks, etc. For example, a machine learning algorithm may be generated through machine learning procedures and applied to identification operations. In some implementations, the machine learning algorithm may be directed to identifying an anatomical object 116 and/or a feature of anatomical object 116 within anatomical scene 114. The machine learning algorithm may operate as an identification function that is applied to individual and/or fused imagery to classify anatomical object 116 in the imagery. The artificial intelligence algorithms may be included in property value system 102 and/or a separate system communicatively coupled with property value system 102. In some instances, the artificial intelligence algorithms may include reference data representative of one or more previously identified anatomical objects 116 and/or one or more features of anatomical objects 116. In instances where the artificial intelligence algorithms include reference data, the image of anatomical scene 114 may be compared to the reference data such as to identify anatomical object 116 and/or a feature of anatomical object 116.

Still other suitable methods may be used for identifying the reference location and/or the additional location in addition to or instead of machine learning algorithms. For example, property value system 102 may be configured to identify the reference location and/or the additional location by implementing and applying object recognition algorithms and image processing algorithms. For example, an object recognition algorithm may be used to identify objects (e.g., anatomical object 116) of predetermined types within the image data received from imaging device 104, such as by comparing the image data received from imaging device 104 to model object data of predetermined types of objects. Such model object data may be stored within a model database that may be communicatively coupled with property value system 102.

Property value system 102 may, at operation 208, determine a value of a property of anatomical object 116 at the additional location relative to the reference location. The value of the property may include any suitable quantitative property of the anatomical object that may be useful in performing the medical procedure. As an illustrative example, the determining the value of the property may include determining a fluorescence value representative of a normalized amount of fluorescence emitted at the additional location relative to a normalized amount of fluorescence emitted at the reference location. Additionally or alternatively, the determining the value of the property may include determining an oxygen saturation value representing an amount of oxygen saturation of anatomical object 116 at the additional location relative to an amount of oxygen saturation of anatomical object 116 at the reference location. Still values of other properties (e.g., temperature, pH, tissue-specific binding, etc.) of the anatomical object may be determined by the property value system. The value of the property may be represented by any suitable metric, such as a discrete value (e.g., a ratio, a percentage, etc.) representative of the property at the additional location relative to the reference location. In some instances, the value of the property of anatomical object 116 at the additional location may be relative to behavior of the property at the reference location over time (e.g., relative to a maximum intensity, a maximum flow, injection timings, etc.).

Property value system 102 may, at operation 210, cause display of output data representative of the reference location, the additional location, and the value of the property of anatomical object 116 at the additional location relative to the reference location. For example, the output data may be displayed by user interface 106 (e.g., display device 118) together with the image of anatomical scene 114. In some implementations, the reference location and/or the additional location may be represented by a virtual overlay (e.g., encircled, highlighted, etc.) on the image of anatomical scene 114 at the reference location and/or the additional location. The value of the property of anatomical object 116 may be displayed as a quantitative value (e.g., as text) and/or as a color map on the image. To illustrate, the color map may include shading associated with the value of the property (e.g., the shading may darken as the value of the property increases and/or the shading may lighten as the value of the property decreases). The color map may additionally or alternatively include different colors and/or color saturation relative to the value of the property.

In some implementations, the causing display of the output data may include updating the output data in real time as the value of the property is updated, such as during the medical procedure. For example, property value system 102 may generate a plurality of images, which may be sequentially output to form a video stream. Additionally or alternatively, the output data may be provided post-processed (e.g., after the medical procedure). This may allow one or more reference locations, additional locations, property values, and/or images to be sampled and stored (e.g., during the course of the medical procedure). Moreover, the post-processed, output data may provide the user with the ability to rewind, review and/or compare the output data at different time points. In some instances, the identifying the reference location, the additional location, and/or causing the display of the output data may be based on one or more characteristics of the medical procedure (e.g., a type of procedure, a specialty associated with the procedure, a patient associated with the procedure, a pre-operative plan associated with the procedure, a preference of a surgeon associated with the procedure, etc.). To illustrate, the reference location may be identified based on a target anatomical object 116 provided by a pre-operative plan for a specific patient.

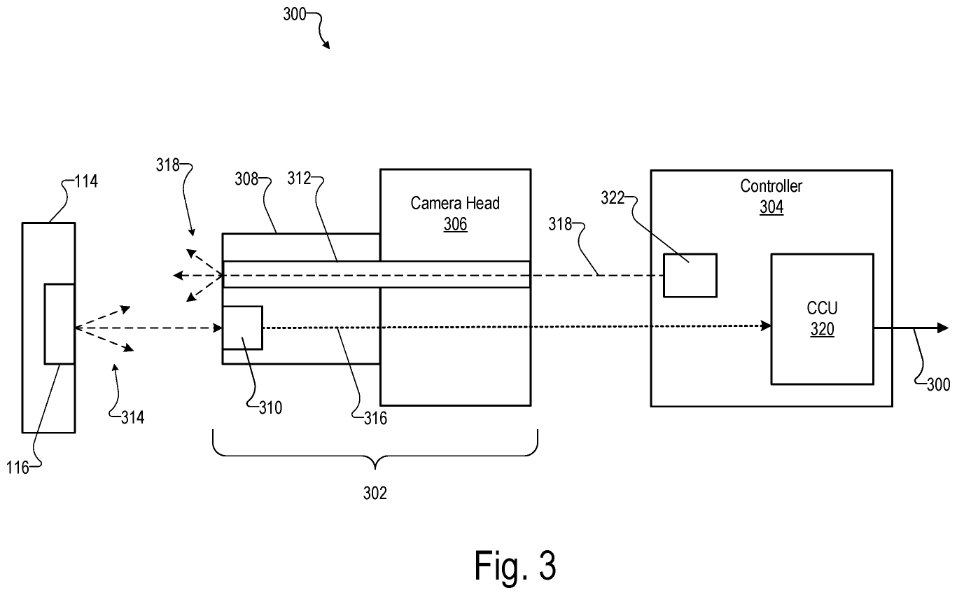

In some implementations, the value of the property provided by property value system 102 may include a fluorescence value associated with a fluorescence emitted from anatomical object 116. For example, FIG. 3 shows an illustrative implementation 300 of a fluorescence imaging system that may be configured to detect fluorescence emitted from anatomical object 116. To illustrate, fluorescence imaging system may be configured to capture fluorescence images of anatomical scene 114 including anatomical object 116 and generate fluorescence image data representative of fluorescence images of anatomical scene 114. As used herein, “fluorescence images” refers to images generated based on detected fluorescence and includes images generated based only on detected fluorescence as well as images generated based on both detected visible light and detected fluorescence (e.g., a visible light image augmented with fluorescence images (an “augmented image”)).

As shown, fluorescence imaging system 300 includes an imaging device 302 and a controller 304. Fluorescence imaging system 300 may include additional or alternative components as may serve a particular implementation, such as various optical and/or electrical signal transmission components (e.g., wires, lenses, optical fibers, choke circuits, waveguides, cables, etc.). While fluorescence imaging system 300 shown and described herein is a fluorescence imaging system, fluorescence imaging system 300 may alternatively include a fluorescence imaging system integrated with a visible light imaging system (e.g., imaging device 104) configured to capture visible light images of the scene. For example, the fluorescence imaging system and visible light imaging system may be physically integrated into the same physical components, or a standalone fluorescence imaging system may be inserted into an assistance port of a visible light endoscope.

Imaging device 302 may be implemented by any suitable device configured to capture fluorescence images of anatomical scene 114. In some examples, as shown in FIG. 3, imaging device 302 is implemented by an endoscope. Imaging device 302 includes a camera head 306, a shaft 308 coupled to and extending away from camera head 306, a fluorescence detection sensor 310, and an illumination channel 312.

Imaging device 302 may be manually handled and controlled (e.g., by a surgeon performing a surgical procedure on a patient). Alternatively, camera head 306 may be coupled to a manipulator arm of a computer-assisted surgical system and controlled using robotic and/or teleoperation technology. The distal end of shaft 308 may be positioned at or near anatomical scene 114 that is to be imaged by imaging device 302. For example, the distal end of shaft 308 may be inserted into a patient.

Fluorescence detection sensor 310 may be implemented by any suitable imaging sensor (e.g., a CCD image sensor, a CMOS image sensor, an InGaAs sensor, etc.) configured to detect (e.g., capture, collect, sense, or otherwise acquire) fluorescence 314 emitted from anatomical object 116 and convert the detected fluorescence into fluorescence image data 316 representative of one or more fluorescence images. As shown, fluorescence detection sensor 310 is positioned at the distal end of shaft 308. Alternatively, fluorescence detection sensor 310 may be positioned closer to the proximal end of shaft 308, inside camera head 306, or outside imaging device 302 (e.g., inside controller 304). In these alternative configurations, optics included in shaft 308 and/or camera head 306 may convey fluorescence from anatomical scene 114 to fluorescence detection sensor 310. For other imaging modalities, other types of sensors (e.g., a temperature sensor, photodiode, etc.) may be used in addition to or instead of fluorescence detection sensor 310.

Illumination channel 312 may be implemented by one or more optical components (e.g., optical fibers, light guides, lenses, etc.). As will be described below, fluorescence excitation illumination 318 may be provided to anatomical scene 114 by way of illumination channel 312 to illuminate anatomical scene 114.

Controller 304 may be implemented by any suitable combination of hardware and/or software configured to control and/or interface with imaging device 302. For example, controller 304 may be at least partially implemented by property value system 102 and/or a computing device included in a computer-assisted surgical system. Controller 304 includes a camera control unit (“CCU”) 320 and an illumination source 322. Controller 304 may include additional or alternative components as may serve a particular implementation. For example, controller 304 may include circuitry configured to provide power to components included in imaging device 302. In some examples, CCU 320 and/or illumination source 322 are alternatively included in imaging device 302 (e.g., in camera head 306). CCU 320 is configured to receive and process fluorescence image data 316 from fluorescence detection sensor 310.

Illumination source 322 is configured to generate and emit fluorescence excitation illumination 318. Fluorescence excitation illumination 318 travels by way of illumination channel 312 to a distal end of shaft 308, where fluorescence excitation illumination 318 exits to illuminate anatomical scene 114, including anatomical object 116. Fluorescence excitation illumination 318 may include one or more broadband spectra of light or may include one or more discrete wavelengths of light. For other imaging modalities, illumination source 322 may be configured to generate or emit other types of illumination (e.g., incandescent, etc.) in addition to or instead of fluorescence excitation illumination 318.

To capture one or more fluorescence images of anatomical scene 114, controller 304 (or any other suitable computing device) may activate illumination source 322 and fluorescence detection sensor 310. While activated, illumination source 322 emits fluorescence excitation illumination 318, which travels via illumination channel 312 to anatomical scene 114. Fluorescence excitation illumination 318 causes anatomical object 116 (e.g., fluorescent agent present in anatomical object 116) to emit fluorescence 314. Fluorescence detection sensor 310 detects fluorescence 314 and converts the detected fluorescence into fluorescence image data 316 representative of one or more fluorescence images of anatomical scene 114. Fluorescence image data 316 is transmitted via a wired or wireless communication link to CCU 320, which processes (e.g., packetizes and/or formats) fluorescence image data 316 and outputs processed fluorescence image data 316. CCU 320 may transmit processed fluorescence image data 316 to property value system 102 for further processing.

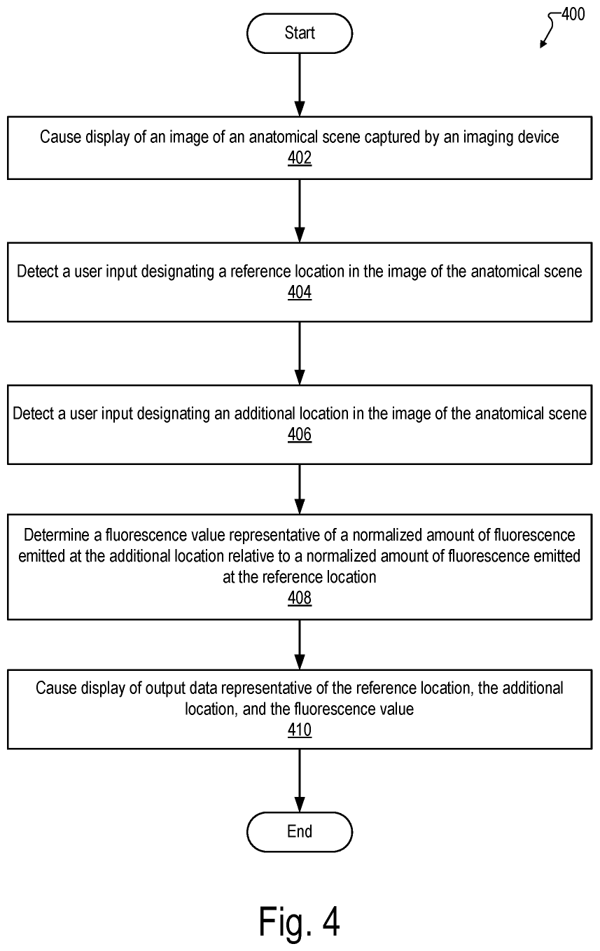

FIG. 4 shows an illustrative method 400 that may be performed by property value system 102 to provide a fluorescence value. While FIG. 4 illustrates exemplary operations according to one embodiment, other embodiments may omit, add to, reorder, and/or modify any of the operations shown in FIG. 4. Moreover, each of the operations depicted in FIG. 4 may be performed in any of the ways described herein.

As shown, property value system 102 may, at operation 402, cause display of an image of anatomical scene 114 as captured by imaging device 104. The causing display of the image may include one or more operations of property value system 102. For example, the causing display of the image may include transmitting the image to user interface 106 for display by display device 118. In some implementations, the causing display of the image may further include receiving an image of anatomical scene 114 captured by imaging device 104, processing the received image, and providing the processed image for display by user interface 106. In some implementations, the image may include or be augmented by a fluorescence image captured by imaging device 302.

Property value system 102 may further, at operation 404, detect a user input designating a reference location in the image of anatomical scene 114 (e.g., associated with anatomical object 116). Property value system 102 may further, at operation 406, detect a user input designating an additional location in the image of anatomical scene 114 (e.g., associated with anatomical object 116).

For example, the image of anatomical scene 114 provided by property value system 102 may be displayed by display device 118 of user interface 106. A user may interact with user input device 120 to designate the reference location and/or the additional location such as by a discrete event (e.g., a touch gesture, a button press, a mouse click, a button release, etc.) on any point of the image of anatomical scene 114 on display device 118. Property value system 102 may be configured to detect the reference location and/or the additional location including one or more points and/or a region of interest on anatomical object 116 depicted in the image of anatomical scene 114.

Property value system 102 may, at operation 408, determine a fluorescence value representative of an amount of fluorescence emitted at the additional location relative to an amount of fluorescence emitted at the reference location. The fluorescence value may be represented by any suitable metric, such as a discrete value (e.g., a percentage, a ratio, etc.) representative of the amount of fluorescence emitted at the additional location relative to the amount of fluorescence emitted at the reference location.

In some implementations, the amount of fluorescence emitted at the reference location and/or the additional location may be based on fluorescence 314 detected by fluorescence detection sensor 310 and/or fluorescence image data 316 outputted by fluorescence detection sensor 310. For example, the image captured by imaging device 104 may include and/or be augmented with fluorescence image data 316 such that the reference location and/or the additional location depicted in the image may be associated with the amount of fluorescence emitted at the reference location and/or the additional location based on fluorescence image data 316.

In some implementations, the amount of fluorescence emitted at the additional location and/or reference location may be normalized, such as to compensate for various factors (e.g., a distribution of fluorescence excitation illumination applied to the anatomy, a position of the light source providing the fluorescence excitation illumination, a depth of the fluorescent agent within the anatomy, etc.) that may affect the amount of fluorescence emitted at the reference location and/or the additional location.

As an illustrative example, the amount of fluorescence emitted at various locations of anatomical object 116 may be a function of the amount of fluorescent agent present at the locations of anatomical object 116, as well as an amount of fluorescence excitation illumination 318 applied to the locations of anatomical object 116. For example, a higher amount of fluorescence emitted at a location of anatomical object 116 may be due to higher amount of fluorescence excitation illumination 318 applied to the location and not necessarily because of a higher amount of the fluorescent agent present at the location.

In some instances, the amount of fluorescence excitation illumination 318 may be non-uniformly distributed across anatomical object 116. To illustrate, an intensity of fluorescence excitation illumination 318 may be higher at a central portion of illumination source 322 and may diminish toward the edges of illumination source 322. Accordingly, the intensity of fluorescence excitation illumination 318 affecting the fluorescent agent (and therefore the amount of fluorescence emitted at the location) may be dependent on the position of the central portion of illumination source 322 relative to the location of anatomical object 116.

Additionally, the intensity of fluorescence excitation illumination 318 affecting the fluorescent agent (and therefore the amount of fluorescence emitted at the location) may be dependent on the position (e.g., distance from the location, angle, etc.) of shaft 308 of imaging device 302. To illustrate, if shaft 308 is moved forward towards and/or backward away from the location of anatomical object 116, the intensity of the fluorescence may vary by increasing with forward motion and/or decreasing with backward motion of shaft 308.

To compensate for such effects, the fluorescence value provided by property value system 102 may be based on a normalized amount of fluorescence emitted at the reference location and/or the additional location. As an example, the normalized amounts of fluorescence may take into consideration excitation falloff of fluorescence excitation illumination 318 across anatomical object 116 and a distance (e.g., a depth) from anatomical object 116 to illumination source 322.

For example, the normalized amount of fluorescence emitted at the reference location may be based on a function of a first reference location quantity representative of an amount of fluorescence (e.g., fluorescence 314) emitted from anatomical scene 114 at the reference location and a second reference location quantity representative of an amount of excitation illumination (e.g., fluorescence excitation illumination 318) incident on the reference location causing the fluorescence to be emitted from anatomical scene 114 at the reference location. Likewise, the normalized amount of fluorescence emitted at the additional location may be based on a function of a first additional location quantity representative of an amount of fluorescence (e.g., fluorescence 314) emitted from anatomical scene 114 at the additional location and a second additional location quantity representative of an amount of excitation illumination (e.g., fluorescence excitation illumination 318) incident on the additional location causing the fluorescence to be emitted from anatomical scene 114 at the additional location. This may allow the fluorescence emitted at the reference location and/or the additional location to be attenuated and/or boosted respectively based on the amount of excitation illumination incident on each of the locations and their depth relative to illumination source 322.

In some implementations, the first reference location quantity representative of the amount of fluorescence emitted at the reference location and/or the first additional location quantity representative of the amount of fluorescence emitted at the additional location may respectively be determined based on fluorescence 314 detected by fluorescence detection sensor 310 and/or fluorescence image data 316 outputted by fluorescence detection sensor 310.

The second reference location quantity representative of the amount of excitation illumination incident on the reference location causing the fluorescence to be emitted at the reference location and/or the second additional location quantity representative of the amount of excitation illumination incident on the additional location causing the fluorescence to be emitted at the additional location may respectively be detected by fluorescence detection sensor 310 and/or another imaging sensor (e.g., a near infrared sensor) configured to detect the amount of excitation illumination incident on the reference location and/or the additional location.

In some implementations, the normalized amounts of fluorescence may be based on a model representing the distribution and/or propagation pattern of fluorescence excitation illumination 318 from illumination source 322 (e.g., to account for excitation falloff of fluorescence excitation illumination 318 across anatomical object 116).

Additionally or alternatively, the normalized amount of fluorescence may be based distance (e.g., a depth) from anatomical object 116 to illumination source 322. For example, property value system 102 may access a depth map that may provide information on intensity variations of fluorescence excitation illumination 318 as a function of spatial separation from illumination source 322 and/or shaft 308. In some implementations, the depth map may be generated such as by processing stereoscopic images, using a simultaneous localization and mapping (SLAM) algorithm, and/or by a depth sensor (e.g., a time of flight sensor) associated with illumination source 322 and/or shaft 308.

Property value system 102 may, at operation 410, cause display of output data representing the reference location, the additional location, and the fluorescence value. For example, the output data may be displayed in user interface 106 (e.g., display device 118) together with the image of anatomical scene 114. In some implementations, the reference location and/or the additional location may be represented by a virtual overlay (e.g., encircled, highlighted, etc.) on the image of anatomical scene 114 at the reference location and/or the additional location. The fluorescence value may further be displayed on the image (e.g., as text, such as near the additional location).

In some implementations, the fluorescence value may be updated in real time as the fluorescence value is updated (e.g., as fluorescent agent flows through anatomical object 116), such as during medical procedure. For example, the fluorescence value may be updated as the normalized amount of fluorescence emitted at the reference location and/or the additional location changes. The output data may further be updated in real time as the fluorescence value is updated.

In some implementations, property value system 102 may be configured to associate the reference location and/or the additional location on anatomical object 116 depicted in the image of anatomical scene 114 with a physical location on anatomical object 116. For example, the reference location and/or the additional location in the image of the anatomical scene may be associated with a corresponding physical location on the anatomical object. In some implementations, the reference location and/or the additional location may be updated based on movement of the physical location of the anatomical object, such as by using a SLAM algorithm. This may allow the representation of reference location and/or the additional location on the image displayed in user interface 106 to be updated when the associated physical location of the reference location and/or the additional location on anatomical object 116 moves.

In some implementations, the positions of the reference location and/or the additional location may continue to be updated while the reference location and/or the additional location are outside of the image, such as when the reference location and/or additional location are obstructed or outside a field of view of the imaging device. This may allow the updated position of the reference location and/or the additional location to be shown when the reference location and/or the additional location return in the image.

Additionally or alternatively, the output data may be provided post-processed (e.g., after the medical procedure). For example, the output data may be provided when the fluorescence value is at a select threshold (e.g., a maximum) at the reference location and/or the additional location.

In some implementations, the image of anatomical scene 114 may include and/or be augmented with fluorescence images captured by imaging device 302 for display in user interface 106 (e.g., display device 118). For example, property value system 102 may include false-color fluorescing regions and/or selectively apply a gain to adjust (e.g., increase or decrease) the illumination intensity of the fluorescing regions (e.g., based on the normalized amounts of fluorescence). Property value system 102 may also generate, based on processed fluorescence image data 316, a plurality of fluorescence images, which may be sequentially output to form a fluorescence video stream.

In some implementations, property value system 102 may further be configured to identify the reference location and/or the additional location based on the detected fluorescence 314 and/or the normalized fluorescence determined at various locations of anatomical scene 114. For example, property value system 102 may use artificial intelligence algorithms, such as machine learning algorithms, to select the reference location and/or the additional location. For example, property value system 102 may determine locations of anatomical scene 114 where the detected fluorescence 314 and/or the normalized fluorescence correspond with a select characteristic (e.g., a maximum, a minimum, an average, a threshold, etc.) to identify the reference location and/or the additional location.

As an illustrative example, the reference location may be identified (e.g., by a user input and/or a machine learning algorithm) at a location of anatomical object 116 that may emit a higher amount of fluorescence relative to other areas of anatomical object 116 depicted in the image of anatomical scene 114. In some scenarios, the higher amount of fluorescence may correspond to a higher amount of tissue perfusion (e.g., depicted by the fluorescent agent flowing through anatomical object 116), which may represent healthy tissue of anatomical object 116. The additional location may also be identified (e.g., by a user input and/or a machine learning algorithm) on anatomical object 116 depicted in the image of anatomical scene 114.

Property value system 102 may determine the fluorescence value based on the amount of normalized fluorescence emitted at the additional location relative to the reference location. Accordingly, the fluorescence value may quantitatively indicate whether the fluorescence emitted at the additional location is higher and/or lower than the fluorescence emitted at the reference location. In some scenarios, the fluorescence value may correspond to an amount of tissue perfusion at the additional location relative to the reference location, which may represent a health of the tissue of anatomical object 116 at the additional location relative to the reference location. In some instances, this may be helpful in determining portions of anatomical object 116 that may be removed.

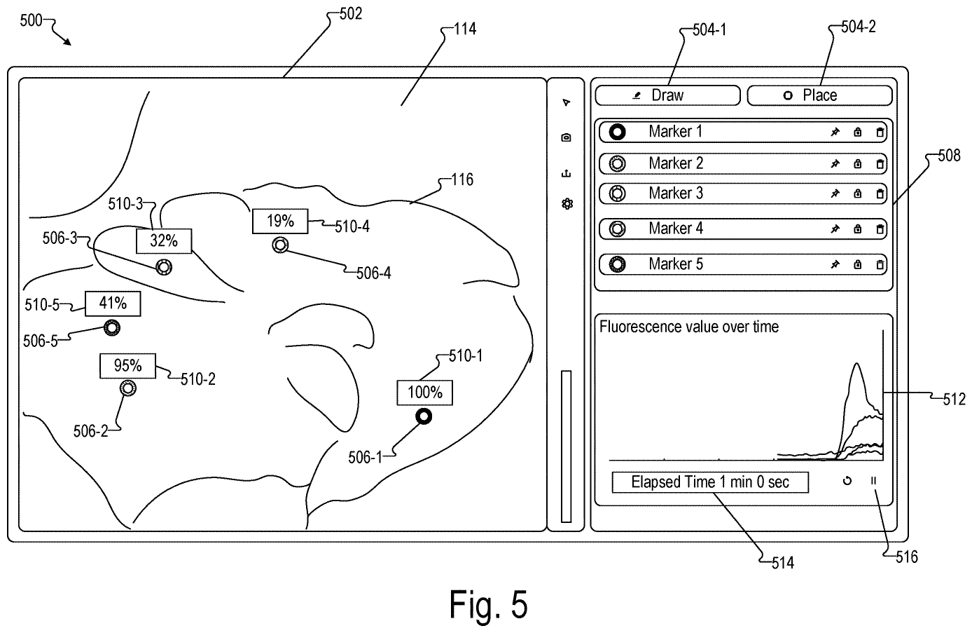

To illustrate, FIG. 5 shows an example user interface view 500 that may display (e.g., by display device 118 of user interface 106) the output data provided by property value system 102. As shown, user interface view 500 may present an image 502 of anatomical scene 114 (e.g., as captured by imaging device 104) that includes a depiction of anatomical object 116. In some implementations, image 502 may include or be augmented by a fluorescence image (e.g., as captured by imaging device 302).

User interface view 500 may further include one or more selectable options 504 (e.g., selectable options 504-1 to 504-2) that may be selected by a user to designate one or more reference locations and/or additional locations on anatomical object 116 depicted in image 502. For example, the user may interact with user input device 120 to perform a discrete event (e.g., a touch gesture, a button press, a mouse click, a button release, etc.) on any point of image 502 to designate locations on image 502.

In some implementations, user interface view 500 may present a first selectable option 504-1 (e.g., “Draw”) that may allow the user to draw one or more shapes (e.g., a point, a circle, a square, a spline, a free-form shape, etc.) on image 502 to designate the one or more reference locations and/or additional locations on anatomical object 116 depicted in image 502. Additionally or alternatively, user interface view 500 may present a second selectable option 504-2 (e.g., “Place”) that may allow the user to place predefined shapes (e.g., circles, squares, triangles, rectangles, etc.) on image 502 to designate the one or more reference locations and/or additional locations on anatomical object 116 depicted in image 502.

For example, FIG. 5 shows a plurality of points 506 (e.g., points 506-1 to 506-5) designated on anatomical object 116 depicted in image 502. Each point 506 may be identified as a reference location or an additional location. For example, a first point 506-1 designated on image 502 may be identified as the reference location and the additional points (e.g., points 506-2 to 506-5) designated on image 502 may be identified as the additional locations. In the illustrated implementation, each point 506 is represented by a marker on image 502 presented within user interface view 500. In some implementations, each marker may have a unique characteristic (e.g., color, pattern, shading, etc.) that may differentiate points 506 on image 502. User interface view 500 may additionally include a legend 508 that may associate each marker with a respective point 506 on image 502.

In some implementations, user interface view 500 may further present a confidence map (e.g., a scale of colors, shading, patterns, etc.) representative of a confidence of the fluorescence image and/or the fluorescence value. For example, an area of image 502 having a low confidence associated with the fluorescence image and/or the fluorescence value may be demarcated as a low confidence area (e.g., the area may include hash lines, gray coloring, etc.) such as to indicate the low confidence associated with the fluorescence image and/or the fluorescence value. In some instances, a confidence value, such as a discrete value (e.g., a percentage, a ratio, etc.), representative of the confidence of the fluorescence image and/or the fluorescence value may be determined. In such instances where a confidence value is determined, the confidence map may be based on a confidence value threshold such that the confidence map may include a first color for confidence values above the confidence value threshold and a second color for confidence values below the confidence value threshold. Additionally or alternatively, property value system 102 may provide a notification and/or prevent points (e.g., points 506) from being designated or interacted with in areas of image 502 having a low confidence of the fluorescence image and/or the fluorescence value (e.g., a confidence value below the confidence value threshold).

User interface view 500 may further present fluorescence values 510 (e.g., fluorescence values 510-1 to 510-5) associated with the reference location and additional locations identified at points 506. To illustrate, if the first point 506-1 is identified as the reference location, a first fluorescence value 510-1 (e.g., “100%”) associated with the first point 506-1 may be presented within user interface view 500. Likewise, additional fluorescence values 510 (e.g., “95%” at second point 506-2, “32%” at third point 506-3, “19%” at fourth point 506-4, and “41%” at fifth point 506-5) representative of the amount of normalized fluorescence emitted at each additional point 506 relative to the first point 506-1 may be presented within user interface view 500. In some implementations, fluorescence values 510 may be presented on image 502 of user interface view 500 (e.g., adjacent to the respective points 506). Additionally or alternatively, fluorescence values 510 may be presented within any other portion of user interface view 500 (e.g., in legend 508).

In some implementations, the output data provided by property value system 102 may further include a plot 512 of fluorescence values 510 over time such that plot 512 may also be presented within user interface view 500. For example, once a point 506 has been identified on image 502, plot 512 may display an intensity of the fluorescence value 510 associated with that point 506 over time. In some implementations, plot 512 may be updated to display fluorescence values 510 in real time (e.g., as fluorescence values 510 are determined and/or updated by property value system 102). In some instances, a point 506 may be deleted from image 502 such that the fluorescence value 510 and/or the intensity of the fluorescence value 510 associated with the point 506 may be removed from plot 512 and/or user interface view 500. Alternatively, in some instances, a point 506 may be moved to a new location on image 502 such that the fluorescence value 510 and/or the intensity of the fluorescence value 510 associated with the point 506 may be removed and restarted (e.g., at the new location) within plot 512 and/or user interface view 500.

In some implementations, plot 512 may further include an elapsed time value 514 that may represent an amount of time that has elapsed (e.g., since the medical procedure began, since the fluorescent agent was administered and/or detected, since fluorescence values 510 began being determined, etc.). In some implementations, user interface view 500 may include an additional selectable option 516 that may allow the user to pause and/or restart plot 512 and/or fluorescence values 510 at a select elapsed time value 514. To illustrate, plot 512 and/or fluorescence values 510 may be paused when the intensity of fluorescence values 510 is at a maximum. Additionally or alternatively, plot 512 may be replayed (e.g., to view previous points in time). Still other suitable implementations for user interface view 500 may be used. For example, plot 512 may adjust for an additional inflow of fluorescence (e.g., from an additional administration of one or more fluorescent agents).

While user interface view 500 includes a 2D image 502 of anatomical object 116, user interface view 500 may additionally or alternatively include a 3D image of anatomical object 116. In a 3D image, points 506 may be designated using 3D interaction (e.g., a 3D cursor, a laser pointer, etc.).

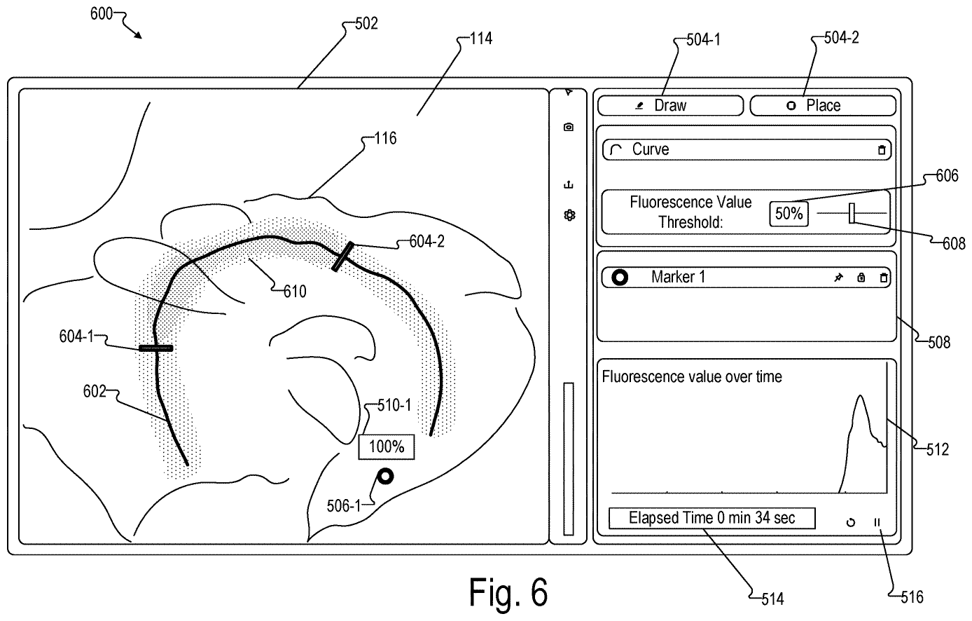

For example, FIG. 6 shows another illustrative user interface view 600 that may display (e.g., by display device 118 of user interface 106) the output data provided by property value system 102. As shown, user interface view 600 includes a first point 506-1 and a region of interest 602. To illustrate, region of interest 602 may include a spline over a portion of image 502 depicting anatomical object 116. For example, a user may interact with user input device 120 to select one or more selectable options 504 to designate first point 506-1 and region of interest 602. In some implementations, first point 506-1 and/or region of interest 602 may include an area on anatomical object 116 that may be depicted in image 502. Additionally or alternatively, first point 506-1 and/or region of interest 602 may include an area in image 502 that may be associated with anatomical object 116. In the illustrated implementation, first point 506-1 is identified as the reference location and region of interest 602 is identified as the additional location. While the reference location is shown in FIG. 6 as being separate from region of interest 602, the reference location may alternatively be included within region of interest 602. Moreover, in instances where image 502 includes a 3D image, points 506 and/or region of interest 602 may be projected onto a surface of anatomical object 116 at depth.

User interface view 600 may further present one or more transition positions 604 (e.g., transition positions 604-1 to 604-2) representative of position in region of interest 602 at which at least some of the fluorescence values transition across a fluorescence value threshold 606 (e.g., “50%”). Fluorescence value threshold 606 may be represented by any suitable metric, such as a discrete value (e.g., a ratio, a percentage, etc.) that may correspond to the fluorescence values. In some implementations, fluorescence value threshold 606 may be identified based on a user input designating fluorescence value threshold 606. For example, user interface view 600 may include an additional selectable option 608 that a user may select (e.g., by interacting with user input device 120) to designate fluorescence value threshold 606. To illustrate, additional selectable option 608 may include a slider that a user may translate in a first direction (e.g., right, up, etc.) to increase fluorescence value threshold 606 and/or in a second direction (e.g., left, down, etc.) to decrease fluorescence value threshold 606. Still other suitable implementations (e.g., typed text, drop-down menu, etc.) of additional selectable option 608 may be used. Additionally or alternatively, fluorescence value threshold 606 may be based on a predetermined threshold. The predetermined threshold may be based on one or more characteristics of the medical procedure (e.g., a type of procedure, a specialty associated with the procedure, a patient associated with the procedure, a pre-operative plan associated with the procedure, a preference of a surgeon associated with the procedure, etc.). Additionally or alternatively, the predetermined threshold may be based on previous predetermined thresholds (e.g., for the medical procedure).

In some instances, property value system 102 may be configured to determine fluorescence values at positions (e.g., a subset of one or more pixels and/or voxels) within region of interest 602 (e.g., along the spline) relative to the reference location (e.g., first point 506-1). To illustrate, the fluorescence values may be representative of the normalized amount of fluorescence emitted at positions within region of interest 602 relative to the normalized amount of fluorescence emitted at the reference location. In some implementations, each position within region of interest 602 may include an area (e.g., a group of pixels and/or voxels) within region of interest 602 such that a combination (e.g., an average, a mean, a median, etc.) of normalized amounts of fluorescence emitted at points within the area may be used to determine the normalized amount of fluorescence emitted at the position.

Property value system 102 may further be configured to determine, based on the fluorescence values at the positions within region of interest 602, the one or more transition positions 604 representing a transition of the fluorescence values across fluorescence value threshold 606. For example, transition positions 604 may include positions within region of interest 602 where the fluorescence values fall below and/or exceed fluorescence value threshold 606.

In some implementations, the fluorescence values may transition across (e.g., fall below and/or exceed) fluorescence value threshold 606 at multiple instances within region of interest 602 (e.g., along the spline). In such instances, a transition position 604 may be determined at a position in region of interest 602 at which at least some of the fluorescence values transition across fluorescence value threshold 606. To illustrate, transition position 604 may be determined at a position representative of a first instance where the fluorescence values cross fluorescence value threshold 606. Additionally or alternatively, transition position 604 may be determined by grouping together multiple instances where the fluorescence values transition across fluorescence value threshold 606 within a distance of each other.

In some implementations, the one or more transition positions 604 may be represented within user interface view 600, such as by one or more markers. For example, FIG. 6 shows transitions positions 604 represented as lines oriented substantially perpendicular relative to region of interest 602, though any other suitable type of markers and/or orientation of markers may be used. To illustrate, region of interest 602 may, in some instances, include an area within a shape (e.g., a circle, a square, hand drawn, etc.) such that transition positions 604 may include a sub-region (e.g., having a shape such as a circle, a square, hand drawn, etc.) within region of interest 602. In some implementations, property value system 102 may apply a function (e.g., a smoothing function) to transition positions 604. While two transition positions 604 are shown in the illustrated implementation of user interface view 600, other implementations may include more or less transition positions 604 (e.g., depending on the number of instances the fluorescence values transition across fluorescence value threshold 606). For example, transition positions 604 may include a plurality of transition positions 604 representative of positions in region of interest 602 at which at least some of the fluorescence values transition across fluorescence value threshold 606.

In some implementations, user interface view 600 may further present a color map 610 (e.g., a scale of colors, shading, patterns, etc.) representative of the fluorescence values provided by property value system 102. To illustrate, a position associated with a higher fluorescence value may be represented as darker and/or lighter on color map 610 than another position associated with a lower fluorescence value. In some implementations, color map 610 may be implemented at positions within region of interest 602, an area including a width around region of interest 602, an area designated by a user, and/or an entirety of image 502. Additionally or alternatively, color map 610 may be based on fluorescence value threshold 606 such that color map 610 may include a first color for fluorescence values above fluorescence value threshold 606 and a second color for fluorescence values below fluorescence value threshold 606. As another example, the fluorescence values may be based on one or more flow rates (e.g., an inflow rate and/or an outflow rate of the fluorescence and/or the fluorescent agent within image 502) such that color map 610 may be representative of the one or more flow rates. To illustrate, a position associated with a higher flow rate may be represented as darker and/or lighter on color map 610 than another position associated with a lower flow rate.

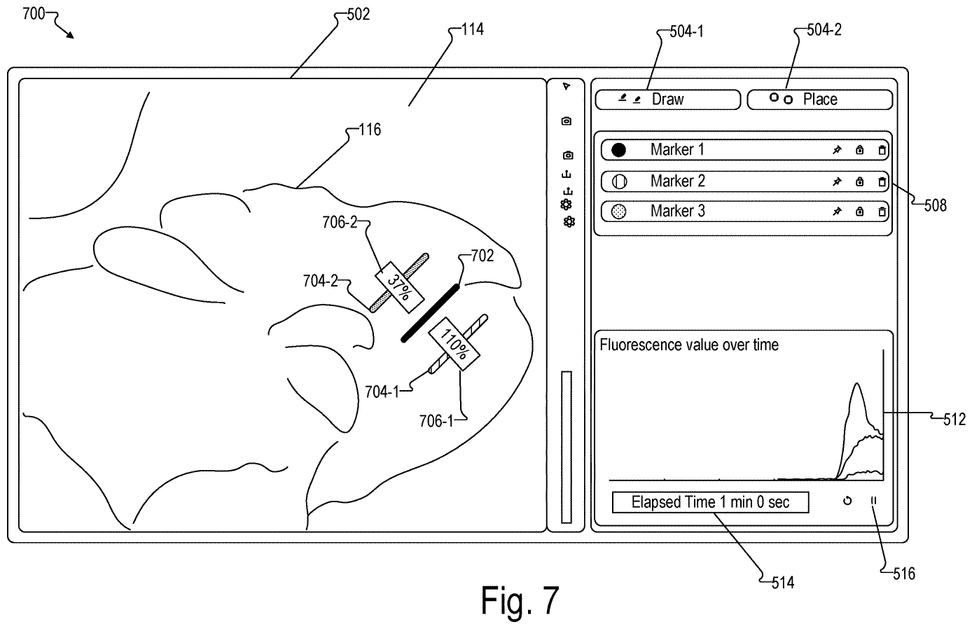

FIG. 7 shows another illustrative user interface view 700 that may display (e.g., by display device 118 of user interface 106) the output data provided by property value system 102. For example, user interface view 700 may present a reference location 702 in image 502 and one or more additional locations 704 (e.g., locations 704-1 to 704-2) spaced a distance away from reference location 702. To illustrate, a user may designate reference location 702 such as by interacting with user input device 120 to select one or more selectable options 504. In the illustrated implementation, reference location 702 is identified as a region having a shape (e.g., rectangular, etc.).

Property value system 102 may be configured to identify the one or more additional locations 704 at a select distance away from reference location 702. The select distance may include a distance in image 502 and/or a distance in physical space (e.g., measured on anatomical object 116). In some implementations, the select distance may be designated based on a predetermined distance and/or a detected user input. The select distance from reference location 702 may be equal for each additional location 704 and/or the select distance from reference location 702 may vary between each additional location 704. In the illustrated implementation, user interface view 700 shows a first additional location 704-1 and a second additional location 704-2 positioned equidistant from reference location 702 on opposing sides of reference location 702.

Each additional location 704 may be represented on image 502 presented within user interface view 700 by a marker. For example, user interface view 700 shows each additional location 704 represented as a marker having a region sized and/or shaped to correspond with reference location 702 and positioned at an orientation substantially parallel to reference location 702, though any other suitable sizes and/or orientations for additional locations 704 may be used. In some implementations, each marker may have a unique characteristic (e.g., color, pattern, shading, etc.) that may differentiate each additional location 704 on image 502. User interface view 700 may additionally include legend 508 that may associate each marker with a respective additional location 704 on image 502.

User interface view 700 may further present fluorescence values 706 (e.g., fluorescence values 706-1 to 706-2) associated with additional locations 704. For example, first fluorescence value 706-1 (e.g., “110%”) may represent the normalized amount of fluorescence emitted at first additional location 704-1 relative to reference location 702 and second fluorescence value 706-2 (e.g., “37%”) may represent the amount of normalized fluorescence emitted at second additional location 704-2 relative to reference location 702. In some instances, additional locations 704 may include an area (e.g., a group of pixels and/or voxels) of image 502 such that a combination (e.g., an average, a mean, a median, etc.) of normalized amounts of fluorescence emitted at points within the area may be used to determine the normalized amount of fluorescence emitted at additional locations 704. Fluorescence values 706 may be displayed within user interface view 700 (e.g., near each additional location 704 on image 502 and/or in legend 508).

While the above examples are directed to user interface views presenting fluorescence values provided by property value system 102, property value system 102 may additionally or alternatively determine values of other properties (e.g., oxygen saturation, temperature, potential hydrogen (pH), tissue-specific binding, etc.) of anatomical object 116 for display in the user interface views.

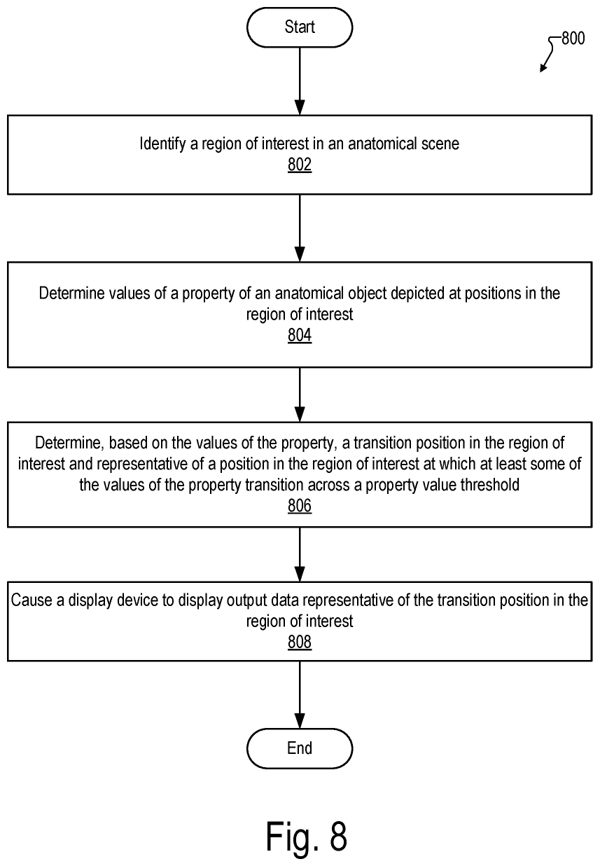

For example, FIG. 8 shows another illustrative method 800 that may be performed by property value system 102 to determine a value of a property (e.g., fluorescence, oxygenation, etc.) of anatomical object 116. While FIG. 8 illustrates exemplary operations according to one embodiment, other embodiments may omit, add to, reorder, and/or modify any of the operations shown in FIG. 8. Moreover, each of the operations depicted in FIG. 8 may be performed in any of the ways described herein.

As shown, property value system 102 may, at operation 802, determine a region of interest (e.g., region of interest 602) in an anatomical scene (e.g., anatomical scene 114). For example, the anatomical scene may be captured by an imaging device (e.g., imaging device 104), such as during a medical procedure. The anatomical scene may depict an anatomical object (e.g., anatomical object 116) such that the region of interest may be associated with the anatomical object. In some implementations, the region of interest may be identified by detecting a user input designating the region of interest and/or using a machine learning algorithm.

Property value system may further, at operation 804, determine values of a property of anatomical object 116 depicted at positions in the region of interest. In some implementations, the determining the values of the property of anatomical object 116 may include determining fluorescence values at positions in the region of interest. For example, the fluorescence values may represent normalized amounts of fluorescence emitted at the positions relative to a normalized amount of fluorescence emitted at a reference location. Additionally or alternatively, the determining the values of the property of the anatomical object may include determining oxygen saturation values at positions in the region of interest.

Property value system 102 may further, at operation 806, determine, based on the values of the property at the positions in the region of interest, a transition position (e.g., transition position 604) representative of a position in the region of interest at which at least some of the values of the property transition across a property value threshold (e.g., fluorescence value threshold 606). The property value threshold may be selected based on a user input designating the property value threshold and/or based on a predetermined property value threshold. In some implementations, the determining the transition position may include updating the transition position in real time as the values of the property of anatomical object 116 at positions in the region of interest change.

Property value system 102 may further, at operation 808, provide output data representative of the transition position in the region of interest. For example, property value system 102 may provide the output data for display by display device 118, such as within a user interface view (e.g., user interface view 600). In some implementations, the output data may be displayed within the user interface view together with the image of anatomical scene 114 captured by the imaging device (e.g., during a medical procedure). Additionally or alternatively, the user interface view may present a color map representing an intensity of the values of the property of anatomical object 116 at positions in the region of interest.

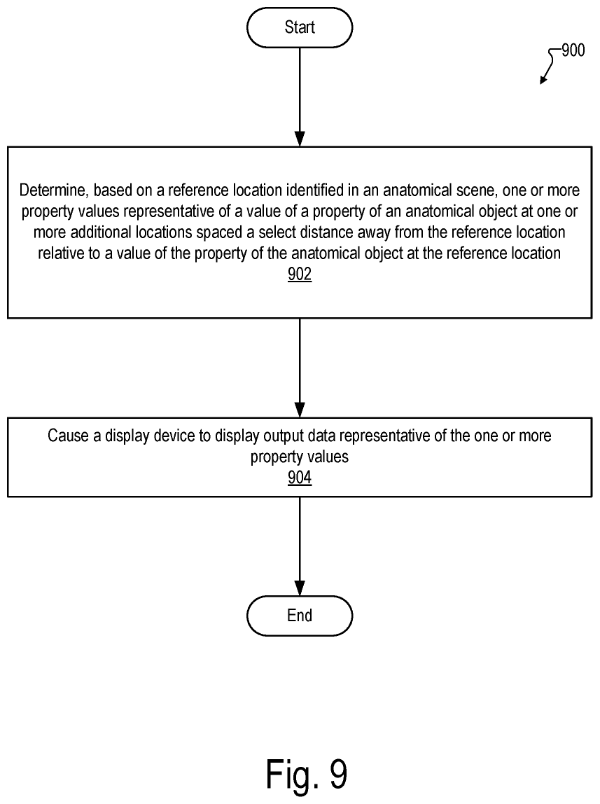

FIG. 9 shows another illustrative method 900 that may be performed by property value system 102. While FIG. 9 illustrates exemplary operations according to one embodiment, other embodiments may omit, add to, reorder, and/or modify any of the operations shown in FIG. 9. Moreover, each of the operations depicted in FIG. 9 may be performed in any of the ways described herein.

As shown, property value system 102 may, at operation 902, determine, based on a reference location (e.g., reference location 702) identified in anatomical scene 114, one or more property values representative of a value of a property of anatomical object 116 at one or more locations (e.g., additional locations 704) spaced a select distance away from the reference location relative to a value of the property of anatomical object 116 at the reference location. For example, the one or more property values may include a first property value representative of a value of the property of anatomical object 116 at a first location (e.g., first additional location 704-1) and a second property value representative of a value of the property of anatomical object 116 at a second location (e.g., second additional location 704-2) such that the first location and the second location are positioned equidistant from the region of interest.

In some implementations, the reference location may be identified in an image (e.g., image 502) of anatomical scene 114 captured by an imaging device (e.g., imaging device 104), such as during a medical procedure. The reference location may be identified by detecting a user input designating the reference location and/or using a machine learning algorithm.

In some implementations, the determining the one or more property values may include determining one or more fluorescence values representative of a normalized amount of fluorescence emitted at the one or more locations relative to a normalized amount of fluorescence emitted at the reference location. Additionally or alternatively, the determining the one or more property values may include determining one or more oxygen saturation values representative of an amount of oxygen saturation at the one or more locations relative to an amount of oxygen saturation at the reference location. In some implementations, the determining the one or more property values may include updating the one or more property values in real time as the value of the property of anatomical object 116 at the one or more locations is updated.

Property value system 102 may further, at operation 904, cause a display device to display output data representative of the one or more property values. For example, the output data may be provided to user interface 106 for display by display device 118, such as within a user interface view (e.g., user interface view 700). In some implementations, the causing display of the output data may further include providing the output data for display in the user interface view together with an image (e.g., image 502) of anatomical scene 114 captured by an imaging device (e.g., imaging device 104), such as during a medical procedure. Additionally or alternatively, the output data may include a plot of the one or more property values over time.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

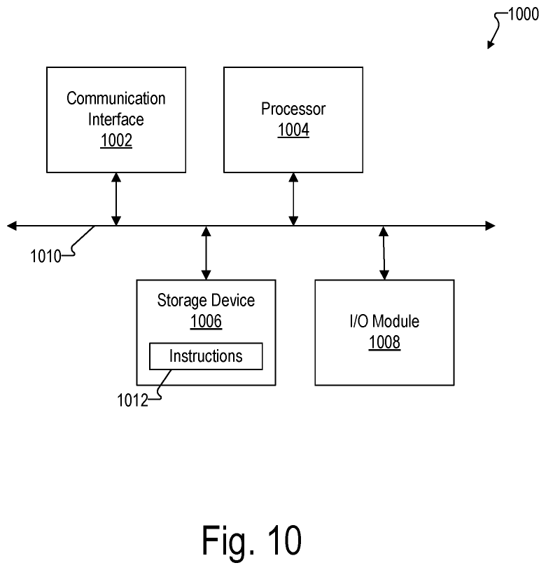

FIG. 10 shows an illustrative computing device 1000 that may be specifically configured to perform one or more of the processes described herein. Any of the systems, computing devices, and/or other components described herein may be implemented by computing device 1000.

As shown in FIG. 10, computing device 1000 may include a communication interface 1002, a processor 1004, a storage device 1006, and an input/output (“I/O”) module 1008 communicatively connected one to another via a communication infrastructure 1010. While an illustrative computing device 1000 is shown in FIG. 10, the components illustrated in FIG. 10 are not intended to be limiting. Additional or alternative components may be used in other embodiments. Components of computing device 1000 shown in FIG. 10 will now be described in additional detail.

Communication interface 1002 may be configured to communicate with one or more computing devices. Examples of communication interface 1002 include, without limitation, a wired network interface (such as a network interface card), a wireless network interface (such as a wireless network interface card), a modem, an audio/video connection, and any other suitable interface.

Processor 1004 generally represents any type or form of processing unit capable of processing data and/or interpreting, executing, and/or directing execution of one or more of the instructions, processes, and/or operations described herein. Processor 1004 may perform operations by executing computer-executable instructions 1012 (e.g., an application, software, code, and/or other executable data instance) stored in storage device 1006.

Storage device 1006 may include one or more data storage media, devices, or configurations and may employ any type, form, and combination of data storage media and/or device. For example, storage device 1006 may include, but is not limited to, any combination of the non-volatile media and/or volatile media described herein. Electronic data, including data described herein, may be temporarily and/or permanently stored in storage device 1006. For example, data representative of computer-executable instructions 1012 configured to direct processor 1004 to perform any of the operations described herein may be stored within storage device 1006. In some examples, data may be arranged in one or more databases residing within storage device 1006.

I/O module 1008 may include one or more I/O modules configured to receive user input and provide user output. I/O module 1008 may include any hardware, firmware, software, or combination thereof supportive of input and output capabilities. For example, I/O module 1008 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1008 is configured to provide graphical data to a display for presentation to a user. The graphical data may be representative of one or more graphical user interfaces and/or any other graphical content as may serve a particular implementation.

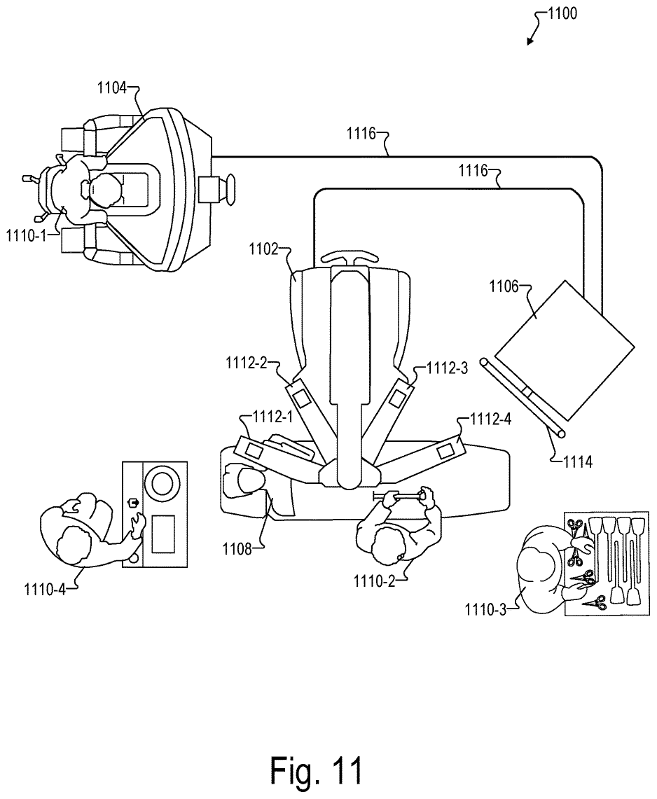

FIG. 11 shows an illustrative computer-assisted medical system 1100 that may be used to perform various types of medical procedures including surgical and/or non-surgical procedures. One or more components of computer-assisted medical system 1100 may be configured to perform one or more of the operations described herein.

As shown, computer-assisted medical system 1100 may include a manipulator assembly 1102 (a manipulator cart is shown in FIG. 11), a user control apparatus 1104, and an auxiliary apparatus 1106, all of which are communicatively coupled to each other. Computer-assisted medical system 1100 may be utilized by a medical team to perform a computer-assisted medical procedure or other similar operation on a body of a patient 1108 or on any other body as may serve a particular implementation. As shown, the medical team may include a first user 1110-1 (such as a surgeon for a surgical procedure), a second user 1110-2 (such as a patient-side assistant), a third user 1110-3 (such as another assistant, a nurse, a trainee, etc.), and a fourth user 1110-4 (such as an anesthesiologist for a surgical procedure), all of whom may be collectively referred to as users 1110, and each of whom may control, interact with, or otherwise be a user of computer-assisted medical system 1100. More, fewer, or alternative users may be present during a medical procedure as may serve a particular implementation. For example, team composition for different medical procedures, or for non-medical procedures, may differ and include users with different roles.

While FIG. 11 illustrates an ongoing minimally invasive medical procedure such as a minimally invasive surgical procedure, it will be understood that computer-assisted medical system 1100 may similarly be used to perform open medical procedures or other types of operations. For example, operations such as exploratory imaging operations, mock medical procedures used for training purposes, and/or other operations may also be performed.

As shown in FIG. 11, manipulator assembly 1102 may include one or more manipulator arms 1112 (e.g., manipulator arms 1112-1 through 1112-4) to which one or more instruments may be coupled. In some implementations, manipulator assembly 1102 may be positioned proximate to a patient 1108 (e.g., as a patient side cart) for the performance of a medical procedure. For example, the instruments may be used for a computer-assisted medical procedure on patient 1108 (e.g., in a surgical example, by being at least partially inserted into patient 1108 and manipulated within patient 1108). While manipulator assembly 1102 is depicted and described herein as including four manipulator arms 1112, it will be recognized that manipulator assembly 1102 may include a single manipulator arm 1112 or any other number of manipulator arms as may serve a particular implementation. While the example of FIG. 11 illustrates manipulator arms 1112 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 1112 shown in FIG. 11. In some implementations, manipulator assembly 1102 may be considered a robotic system that is a component of computer-assisted medical system 1100.

During the medical operation, user control apparatus 1104 may be configured to facilitate teleoperational control by user 1110-1 of manipulator arms 1112 and instruments attached to manipulator arms 1112. To this end, user control apparatus 1104 may provide user 1110-1 with imagery of an operational area associated with patient 1108 as captured by an imaging device. To facilitate control of instruments, user control apparatus 1104 may include a set of master controls. These master controls may be manipulated by user 1110-1 to control movement of the manipulator arms 1112 or any instruments coupled to manipulator arms 1112.

Auxiliary apparatus 1106 may include one or more computing devices configured to perform auxiliary functions in support of the medical procedure, such as providing insufflation, electrocautery energy, illumination or other energy for imaging devices, image processing, or coordinating components of computer-assisted medical system 1100. In some examples, auxiliary apparatus 1106 may be configured with a display monitor 1114 configured to display one or more user interfaces, or graphical or textual information in support of the medical procedure. In some instances, display monitor 1114 may be implemented by a touchscreen display and provide user input functionality. Augmented content provided by a region-based augmentation system may be similar, or differ from, content associated with display monitor 1114 or one or more display devices in the operation area (not shown).

Manipulator assembly 1102, user control apparatus 1104, and auxiliary apparatus 1106 may be communicatively coupled one to another in any suitable manner. For example, as shown in FIG. 11, manipulator assembly 1102, user control apparatus 1104, and auxiliary apparatus 1106 may be communicatively coupled by way of control lines 1116, which may represent any wired or wireless communication link as may serve a particular implementation. To this end, manipulator assembly 1102, user control apparatus 1104, and auxiliary apparatus 1106 may each include one or more wired or wireless communication interfaces, such as one or more local area network interfaces, Wi-Fi network interfaces, cellular interfaces, and so forth.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
one or more processors communicatively coupled to the memory and configured to execute the instructions to perform a process comprising:
causing display, in a user interface during a medical procedure, of an image of an anatomical scene captured by an imaging device during the medical procedure;
identifying a reference location in the image of the anatomical scene;
identifying a plurality of additional locations in the image of the anatomical scene;
determining fluorescence values representative of normalized amounts of fluorescence emitted at the plurality of additional locations relative to a normalized amount of fluorescence emitted at the reference location;
determining, based on the fluorescence values, one or more transition positions representative of a transition of the fluorescence values across a fluorescence value threshold; and
causing display, in the user interface together with the image of the anatomical scene during the medical procedure, of output data representative of the reference location, the plurality of additional locations, and the one or more transition positions.

2. The system of claim 1, wherein the identifying the plurality of additional locations includes identifying one or more points in the image of the anatomical scene.

3. The system of claim 1, wherein the identifying the plurality of additional locations includes identifying a region of interest in the image of the anatomical scene.

4. The system of claim 1, wherein the identifying the plurality of additional locations includes identifying a first location spaced a select distance away from the reference location.

5. The system of claim 4, wherein the identifying the plurality of additional locations further includes identifying a second location spaced a select distance away from the reference location such that the first location and the second location are positioned equidistant from the reference location.

6. (canceled)

7. The system of claim 1, wherein the fluorescence value threshold is selected based on a designation of the fluorescence value threshold by a user input.

8. The system of claim 1, wherein the identifying one or both of the reference location or the plurality of additional locations includes detecting a designation of the reference location or the plurality of additional locations by a user input.

9. The system of claim 8, wherein the user input is associated with a computer-assisted medical system.

10. The system of claim 1, wherein the identifying one or both of the reference location or the plurality of additional locations includes using a machine learning algorithm.

11. The system of claim 1, wherein the output data includes a plot of the fluorescence values over time.

12. The system of claim 1, wherein the output data includes a color map representative of the fluorescence values.

13. The system of claim 1, wherein the output data includes a confidence map representative of a confidence of the fluorescence values.

14. The system of claim 1, wherein:
the normalized amount of fluorescence emitted at the reference location is based on a function of a first quantity representative of an amount of fluorescence emitted from the anatomical scene at the reference location and a second quantity representative of an amount of excitation causing the fluorescence to be emitted from the anatomical scene at the reference location; and
the normalized amounts of fluorescence emitted at the plurality of additional locations are based on a function of first quantities representative of amounts of fluorescence emitted from the anatomical scene at the additional location and a second quantity representative of an amount of excitation causing the fluorescence to be emitted from the anatomical scene at the plurality of additional locations.

15. The system of claim 1, wherein the process further comprises associating one or both of the reference location or the plurality of additional locations in the image of the anatomical scene with a physical location on an anatomical object.

16. The system of claim 15, wherein the causing the display of the output data includes updating display of a position of one or both of the reference location or the plurality of additional locations based on movement of the physical location on the anatomical object.

17. The system of claim 1, wherein:
the determining the fluorescence values includes updating the fluorescence values in real time as one or both of the normalized amounts of fluorescence emitted at the plurality of additional locations or the normalized amount of fluorescence emitted at the reference location changes; and
the causing display of the output data includes updating display of the output data in real time as the fluorescence values is updated.

18. The system of claim 1, wherein the medical procedure includes a minimally-invasive medical procedure.

19. A method comprising:
causing display, by at least one computing device and in a user interface during a medical procedure, of an image of an anatomical scene captured by an imaging device during the medical procedure;
identifying, by the at least one computing device, a reference location in the image of the anatomical scene;
identifying, by the at least one computing device, a plurality of additional locations in the image of the anatomical scene;
determining, by the at least one computing device, fluorescence values representative of normalized amounts of fluorescence emitted at the plurality of additional locations relative to a normalized amount of fluorescence emitted at the reference location;
determining, by the at least one computing device and based on the fluorescence values, one or more positions representative of a transition of the fluorescence values across a fluorescence value threshold; and
causing display, by the at least one computing device and in the user interface together with the image of the anatomical scene during the medical procedure, of output data representative of the reference location, the plurality of additional locations, and the one or more transition positions.

20. A non-transitory computer-readable medium storing instructions that, when executed, direct a processor of a computing device to:
cause display, in a user interface during a medical procedure, of an image of an anatomical scene captured by an imaging device during the medical procedure;
identify a reference location in the image of the anatomical scene;
identify a plurality of additional locations in the image of the anatomical scene;
determine fluorescence values representative of normalized amounts of fluorescence emitted at the plurality of additional locations relative to a normalized amount of fluorescence emitted at the reference location;
determine, based on the fluorescence values, one or more transition positions representative of a transition of the fluorescence values across a fluorescence value threshold; and
cause display, in the user interface together with the image of the anatomical scene during the medical procedure, of output data representative of the reference location, the plurality of additional locations, and the one or more transition positions.

21.-75. (canceled)
